(19) United States
(12) Patent Application Publication
(10) Pub. No.: US 2018/0160695 A1
(43) Pub. Date: Jun. 14, 2018

(54) USE OF ICE STRUCTURING PROTEIN AFP19 EXPRESSED IN FILAMENTOUS FUNGAL STRAINS FOR PREPARING FOOD

(71) Applicant: DSM IP ASSETS B.V., Heerlen (NL)

(72) Inventors: Petrus Jacobus Theodorus DEKKER, Echt (NL); Jeroen Adrianus Johannes NOOIJENS, Echt (NL)

(21) Appl. No.: 15/578,342

(22) PCT Filed: Jun. 1, 2016
(86) PCT No.: PCT/EP2016/062352

§ 371 Date: Nov. 30, 2017

(30) Foreign Application Priority Data
Jun. 2, 2015 (EP) 15170233.9

(51) Int. Cl. A23C 13/08; C12N 9/62; C12N 15/81; C07K 14/39; A23C 13/12; A23C 19/068; A23C 19/072; A23C 19/097; A23L 3/365; A23L 3/375; A23J 1/18

(52) U.S. Cl. CPC A23C 13/085; C12N 9/62; C12N 15/815; C07K 14/39; A23C 13/12; A23J 1/18; A23C 19/072; A23C 19/0976; A23L 3/365; A23L 3/375; A23C 19/0684

(57) ABSTRACT

The present invention relates to a food product which food product is frozen and thawed before consumption as well as to methods for preparing such a food product. The invention further describes production of an ice structuring protein (ISP) in high amounts.

### FIELD OF THE INVENTION

The present invention relates to a food product which food product is frozen and thawed before consumption as well as to methods for preparing such a food product. The invention further describes production of an ice structuring protein (ISP) in high amounts.

### BACKGROUND TO THE INVENTION

An interesting group of proteins that has potentially many application possibilities is ice structuring proteins (ISP), often referred to as antifreeze proteins (AFP).

Warren et al. (U.S. Pat. No. 5,118,792) have suggested adding purified ISPs directly to food products prior to freezing to improve preservation characteristics during frozen storage. WO90/13571 A1 teaches methods of improving the freeze tolerance of food products by suppressing ice crystal growth or inhibiting ice recrystallization using antifreeze polypeptides in isolated form. A wide range of applications for ISP's in food and non-food have been suggested in the past, e.g. exemplified in Griffith and Vanya Ewart (1995) Biotechnology Advances 13:375-402 and EP2602263A2. However, many applications are currently economically not feasible due to the high cost in use of ISP.

Cost in use of such ISP's must be as low as possible to allow development of many different applications. Although the AFP type III HPLC12 from ocean pout is currently used for ice cream production on industrial scale, the difficulties associated with producing ISPs in large quantities at an economic attractive price preclude them from use in other industrial applications. An ideal ISP that can be used in different applications would be highly active at low concentration, low in cost, readily available, and simple to use.

A low cost in use of ISP can be obtained by selecting an ISP that has high ice structuring activity per mol protein. The minimal concentration of the best studied, and industrially used ISP (type III AFP from ocean pout) to obtain recrystallization inhibition in a 30% sucrose solution at −6° C. has been reported to be >700 nM (Smallwood et al (1999) Biochem. J. 340:385-391; Tomczak et al (2003) Biochem. Biophys. Res. Comm. 311: 1041-1046). Consequently, a relatively high concentration of ISP is required in the application to obtain satisfactory results. Unilever has reported that the concentration of ISP (type III AFP) in ice cream application is ˜50 mg/kg (w/w) (Lewis (2006) Application for the Approval of Ice Structuring Protein Type III HPLC 12 Preparation for use in Edible Ices, Regulation (EC) No 258/97 of the European Parliament and of the Council of 27 Jan. 1997 Concerning Novel Foods and Novel Food Ingredients), which is equivalent to 7 μM for this ISP. It has also been reported that ice crystal growth can be inhibited by 3-25 μM type III AFP (Li and Hew (1991) Protein engineering 4:1003-1008). An ISP which gives a similar effect with a lower required dosage would be beneficial for decreasing the cost-in-use of these proteins and would open up the possibility to develop additional applications.

Also high expression of ISP per kg fermentation broth will lead to a reduced cost price and a low cost-in-use. High expression will also lead to a more pure product that only requires a minimum of purification, thus further reducing cost price.

The productivity of ISP's that are currently described in literature is however low. Expression of type III AFP from ocean pout in the bakers' yeast Saccharomyces cerevisiae has been reported to be difficult (U.S. Pat. No. 6,914,043 B1) and only detectable when the culture broth supernatant was undiluted, suggesting a low level of expression.

Also expression of the ISP of Leucosporidium (LelBP) in Escherichia coli or Pichia pastoris is described to be between 2.1 and 61.2 mg per liter culture broth in shake flask (Park et al (2012) Cryobiology 64286-296), despite the track record of both microorganisms to successfully express heterologous proteins at high level.

Recently Lee et al have summarized all literature on the expression of known ISP's and concluded that expression levels do not exceed 175 mg/l (Lee et al (2013) Appl. Microbiol. Biotechnol. 97:3383-3393). They suggest that the application of ISP's is largely hampered by the lack of an economic production systems.

The same authors managed to increase productivity of LelBP to ˜300 mg/l by a combined fed batch and induction of production by addition of methanol at reduced temperature. Due to the low productivity in fermentation, the protein had to be concentrated and purified before it could be used in further experiments, leading to a very poor yield and consequently a high cost price. Such measures may be useful for lab scale fermentation but are not economic for industrial production. Because of this the cost in use of the currently described ISP's is high and therefore the use of ISP in industry is limited.

Besides the cost in use it is also important that the end product has GRAS status (Generally Regarded As Safe) for the application of an ISP in a food product. Many of the ISPs described in literature are expressed in micro-organisms or produced with processes lacking this status and can therefore not be used in food applications. For example LelBP is currently expressed in Pichia pastoris, a yeast which requires the addition of toxic methanol for induction of the expression of LelBP (Lee et al (2013)).

An ISP that is highly active, low in cost, and food-grade is now available. Surprisingly such an ISP has advantages when used in a food product which is frozen and thawed before consumption.

### SUMMARY OF THE INVENTION

The present invention is based on the surprising effect of an ice structuring protein from Leucosporidium sp. (AFP19) produced in a filamentous fungus, when used in a food product which is frozen and completely thawed before consumption. The expression level of AFP19 is, surprisingly, exceptionally high in filamentous fungi, much higher than described in literature for expression in bacteria or yeast.

Moreover, AFP19 as expressed in filamentous fungi shows very good ice re-crystallization inhibition activity at extremely low concentrations. This ISP is shown to be active in ice recrystallization at ˜20 nM, a 35-fold lower concentration than found for the current industry standard type III AFP from ocean pout.

Surprisingly, productivity of AFP19 in a filamentous fungus was higher than 1 g/I at shake flask scale. Productivity of the same protein at shake flask scale in yeast or bacteria has been reported in literature to be a factor 15-500 lower (named LelBP). Therefore the cost in use of AFP19 expressed in filamentous fungi will be much lower than all currently known ISP's. Consequently many more industrial applications may be economically possible using AFP19 expressed in filamentous fungi as compared with currently known ISPs.

Furthermore, it appears that the ISP as produced in a filamentous fungus is a stable protein: possible stabilizing properties are N- or O-glycosylation and/or a block by pyroglutamate at the N-terminus.

According to the invention, there is provided a method for preparing a food product which food product is frozen and completely thawed before consumption (i.e. a method for preparing a frozen food product which frozen food product is completely thawed before consumption or use), which method comprises incorporating an ice structuring protein (ISP) in said food product and freezing the prepared food product (and optionally storing the prepared food product at frozen conditions).

The invention also provides a food product which is frozen and completely thawed before consumption (i.e. a frozen food product which is completely thawed before consumption), wherein said food product comprises an ice structuring protein. Preferably, the frozen food product is obtained by the above described method.

### BRIEF DESCRIPTION OF THE DRAWINGS

FIG. 1: Average size (volume) of cracks (not green not red) in cheeses ripened for different times and frozen at −20° C. for 4 weeks. Each time point (2, 4 or 10 weeks) show 2 bars. The left bar at each time point represents cheeses with added ISP (=AFP19); the right bar at each time point represent cheeses without ISP, i.e. without AFP19.

FIG. 2: Volume of whipped cream. Cream was stored at 4 or −20° C. before whipping. Amount of ISP (=AFP19) addition is indicated.

FIG. 3 shows a physical map of the pGBTOP-16 vector used for cloning of the AFP19 gene. The pGBTOP-16 vector is derived from the pGBTOP-12 vector described in WO2011/009700. In addition to pGBTOP-12, it contains the ccdB gene from E. coli for positive selection for presence of an insert between the EcoRI and PacI cloning sites. The PacI restriction site replaces the SnaBI restriction site present in pGBTOP-12.

FIG. 4 shows LC MS/MS size determination of AFP19 from Aspergillus niger, before and after PNGase F treatment.

FIG. 5 shows the detected masses of AFP forms before and after deglycosylation with PNGase f. C-terminus truncated form of pyroglutamated AFP are indicated in the Figure with grey rectangles. 162 Da represents the mass of a hexose.

FIG. 6 Typical TPA curve during a double bite compression test.

### DESCRIPTION OF THE SEQUENCE LISTING

SEQ ID NO: 1 sets out the protein sequence of the ISP of Leucosporidium (AFP19). This sequence consists of a signal sequence of 20 amino acids for efficient secretion in Leucosporidium and a deduced mature protein sequence of 241 amino acids. The amino acid sequence of AFP19 of Leucosporidium is also set out in Swiss-Prot/TrEMBL (accession number: C7F6X3) and Genbank accession number ACU30807.1.

SEQ ID NO: 2 sets out a codon-adapted DNA sequence for expression of SEQ ID NO: 1 in Aspergillus niger

SEQ ID NO: 3 sets out a codon-adapted DNA sequence for expression of SEQ ID NO: 1 in Aspergillus niger containing additional restriction sites for subcloning in an Aspergillus expression vector.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The present invention describes the expression of an ice structuring protein (ISP), for example that from a Leucosporidium sp., such as AFP19, the full length amino acid sequence of which is set out in SEQ ID NO: 1, in a filamentous fungus. The expression level of such proteins has been demonstrated to be unexpectedly and exceptionally high in filamentous fungi; much higher than expression levels described in literature for bacteria or yeast.

In addition, the ISP (as described herein), as expressed using filamentous fungus, can be distinguished, at the chemical level, from the equivalent protein isolated either from a wild-type source or from an equivalent protein as expressed in bacteria or yeast.

The ISP as described herein is used in the preparation of a food composition which is frozen and (completely) thawed before consumption, i.e. in the preparation of a frozen food which is completely thawed before consumption. A non-limiting example of such a food product is cheese or cream. The invention does therefore not relate to for example an ice cream or a sorbet or frozen yoghurt which products are consumed in a completely frozen state or at least in a partly frozen state.

Use of the ISP as described herein confers a number of advantages either on the final food composition or in the preparation of such a composition.

In relation to the preparation of a food composition, for example, use of an ISP allows slower hardening in the preparation of a frozen food composition, for example in the preparation of frozen cream or frozen cheese. This may allow the use of larger package sizes and/or the use of less power input in blast freezing during hardening.

In relation to a food composition itself as prepared using an ISP as described herein, such a food composition may have an increased shelf life without quality loss (as compared to a corresponding food composition prepared without use of an ISP as described herein). In particular, a food composition prepared using an ISP as described herein may be more resistant to heat shock that a food composition not prepared with such as ISP and it may be possible to store such a food composition at a higher temperature than a food composition not prepared with such an ISP. The quality of such food composition may be less vulnerable to the temperature fluctuations that accompany transport and retail handling of such food product.

Further, the resulting food composition as prepared using an ISP as described herein may have improved textural properties, as will be discussed, for example, in the experimental part herein.

Accordingly, the invention relates to a method for preparing a food product which food product is frozen and completely thawed before consumption (i.e. a method for preparing a frozen food product which is completely thawed before consumption), which method comprises incorporating an ice structuring protein (ISP) in said food product and freezing the prepared food product (and optionally storing the prepared food product at frozen conditions). Alternatively phrased, the invention provides a method for reducing textural defects formation of a food product upon freezing (for example cheese or cream) which method comprises incorporating an ice structuring protein (ISP) in said food product and freezing the prepared food product (and optionally storing the prepared food product at frozen conditions). In more detail, the invention provides a method for reducing textural defects in a food product (or a method for preparing a food product) which is subjected to at least one freeze/complete thaw cycle, which method comprises incorporating an ice structuring protein (ISP) in said food product, freezing the prepared food product (optionally storing the prepared food product at frozen conditions for a certain amount of time) and completely thawing the frozen food product. The texture is compared to a similar frozen/thawed food product which does not comprise an ISP. In yet another alternative phrasing, the invention provides a method for improving the whippability of cream that is subjected to a freeze/complete thaw cycle, which method comprises incorporating an ice structuring protein (ISP) in cream, freezing the cream/ISP mixture (and optionally storing the prepared food product at frozen conditions) and completely thawing the frozen cream. The whippability is improved when compared to a similar frozen/thawed cream which does not comprise an ISP. In yet another aspect, the invention provides a method for preparing frozen whipped cream, which method comprises incorporating an ice structuring protein (ISP) in whipped cream, freezing the whipped cream/ISP product (and optionally storing the prepared food product at frozen conditions).

In a preferred option, any of the above described methods of the invention further comprises complete thawing of the frozen food product. Depending on the type of food product other optional steps are:
in case the food product is cheese: ripening of the cheese before freezing or after completely thawing storing the cheese at a suitable temperature (for example at refrigerator temperatures such as 2-7 degrees Celsius or at room temperature). A preferred order of steps for preparing cheese in the presence of an ISP is: adding an ISP to the cheese production process, ripening of the cheese to the desired maturation (for example young cheese or old cheese), freezing of the cheese after ripening, optionally storing the cheese at frozen conditions (for example, for a period of days to months), completely thawing of the frozen cheese and storing of the completely thawed cheese at a desired temperature (but not in a freezer).
in case the food product is cream: whipping of the cream after complete thawing. A preferred order of steps is adding an ISP to cream, freezing of the cream/ISP mixture, optionally storing the cream/ISP mixture at frozen conditions (for example, for a period of days to months), completely thawing of the frozen cream/ISP mixture and whipping said thawed cream/ISP mixture. Alternatively, the invention also provides a method for preparing whipped cream, which method comprises incorporating an ice structuring protein (ISP) in said cream, whipping the cream (or first whipping the cream and then incorporating the ISP), freezing the cream (optionally storing the whipped frozen cream) and completely thawing the whipped cream.

Irrespective of the produced (frozen) food product, the incorporation of the ISP is obtained by adding the ISP in an effective amount (which can easily be determined by the skilled person) and by taking measures to distribute ISP throughout the food product (for example by mixing).

The invention further provides a food product which is frozen and completely thawed before consumption (i.e. a frozen food product which is completely thawed before consumption), wherein said food product comprises an ice structuring protein. Such a food product is for example obtainable by a method of the invention.

The ISP used in any of the methods of the invention or the ISP present in a (frozen) food product of the invention is preferably produced by using a nucleic acid construct which comprises:
a nucleic acid sequence encoding an ice structuring protein (ISP) comprising the sequence set out in SEQ ID NO: 1 or a sequence at least 80% identical thereto or comprising the sequence set out in amino acids 21 to 261 of SEQ ID NO: 1 or a sequence at least 80% identical thereto; and, linked operably thereto,
control sequences permitting expression of the nucleic acid sequence in a filamentous fungal host cell.

The nucleic acid construct may be incorporated into a vector, such as an expression vector and/or into a host cell in order to effect expression of the ISP.

The term “nucleic acid construct” is herein referred to as a nucleic acid molecule, either single- or double-stranded, which is isolated from a naturally-occurring gene or, more typically, which has been modified to contain segments of nucleic acid which are combined and juxtaposed in a manner which would not otherwise exist in nature. That is to say, a nucleic acid construct used in a method or food product of the invention is a recombinant construct i.e. one which is non-naturally occurring. The term nucleic acid construct is synonymous with the term “expression cassette” when the nucleic acid construct contains all the control sequences required for expression of a coding sequence, wherein said control sequences are operably linked to said coding sequence.

Such a nucleic acid construct comprises a nucleic acid sequence encoding an ice structuring protein (ISP).

For the purposes of this invention, “ice structuring protein (ISP)” or, alternatively, “antifreeze protein (AFP)” or “ice binding protein” refers to a polypeptide capable of binding small ice crystals so as to inhibit growth and recrystallization of ice crystals. Recrystallization inhibition (RI) can be measured as described in Example 4 (see Tomczak et al (2003) Biochem. Biophys. Res. Comm. 311: 1041-1046).

An ISP may also, or alternatively, be a polypeptide which is capable of creating or increasing the difference between the melting point and freezing point of a solution, i.e. is one which is capable of increasing the thermal hysteresis of a solution, in comparison with the same solution not comprising an ISP. Thermal hysteresis may be measured with a Clifton nanolitre osmometer.

The nucleic acid sequence (comprised within a nucleic acid construct as described herein) encodes an ISP comprising:

the amino acid sequence set out in SEQ ID NO: 1 or a sequence at least 80% identical thereto; or

the amino acid sequence set out in amino acids 21 to 261 of SEQ ID NO: 1 or a sequence at least 50%, 60%, 70% or 80% identical thereto.

SEQ ID NO: 1 sets out the protein sequence of the ISP of Leucosporidium (AFP19). This sequence consists of a signal sequence of 20 amino acids for efficient secretion in Leucosporidium and a deduced mature protein sequence of 241 amino acids.

In a nucleic acid construct as used herein, the nucleic acid sequence may encode an ISP comprising the sequence set out in SEQ ID NO: 1 or a sequence at least 85%, at least 90%, at least 95%, at least 98% or at least 99% identical thereto or comprising the sequence set out in amino acids 21 to 261 of SEQ ID NO: 1 or a sequence at least 85%, at least 90%, at least 95%, at least 98% or at least 99% identical thereto.

That is to say, the nucleic acid sequence used in a nucleic acid construct as described herein may share at least 50%, 60%, 70%, 80%, at least 85%, at least 90%, at least 95%, at least 97%, at least 98%, at least 99% sequence identity with either of the amino acid sequence set out in SEQ ID NO: 1 or the amino acid sequence set out in amino acids 21 to 261 of SEQ ID NO: 1.

In a nucleic acid construct as used herein, the nucleic acid may encode an ISP comprising an amino acid sequence obtainable from an arctic yeast of the genus Leucosporidium.

For the purpose of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

The amino acid sequence of the ISP which is actually expressed in a filamentous fungi may not comprise all of those amino acids theoretically encoded by the nucleic acid sequence. For example, the amino acid sequence may be shorter than that theoretically encoded by the nucleic acid sequence, for example in view of amino acids missing from the N- and/or C-terminal ends of the ISP (in comparison with the predicted sequence). For example, the amino acid sequence may be one, two, three, four, five, six, seven, eight, nine, ten, eleven or twelve or more amino acids shorter than the predicted mature sequence of amino acids 21 to 261 of SEQ ID NO: 1. In this case, identity may be calculated on the basis of an alignment which excludes those amino acids theoretically, but not actually, present.

Thus, a nucleic acid sequence (comprised within a nucleic acid construct as used herein) may encode an ISP comprising:

the amino acid sequence set out in amino acids 21 to 260 of SEQ ID NO: 1, amino acids 21 to 259 of SEQ ID NO: 1, amino acids 21 to 258 of SEQ ID NO: 1, amino acids 21 to 257 of SEQ ID NO: 1, amino acids 21 to 256 of SEQ ID NO: 1, amino acids 21 to 255 of SEQ ID NO: 1, amino acids 21 to 254 of SEQ ID NO: 1, amino acids 21 to 253 of SEQ ID NO: 1, amino acids 21 to 252 of SEQ ID NO: 1, amino acids 21 to 251 of SEQ ID NO: 1, amino acids 21 to 250 of SEQ ID NO: 1, amino acids 21 to 249 of SEQ ID NO: 1, amino acids 21 to 248 of SEQ ID NO: 1, amino acids 21 to 247 of SEQ ID NO: 1 or amino acids 21 to 246 of SEQ ID NO: 1 or a sequence at least 50%, at least 60%, at least 70%, at least 80%, at least 85%, at least 90%, at least 95%, at least 98% or at least 99% identical to any one thereto.

In another preferred option, the amino acid sequence of the ISP has been modified resulting in a further improved expression in a filamentous fungi according the method as described in WO2010/102982.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percentage of sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence as used in the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences as used herein can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules as used in the present invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

The nucleic acid sequence encoding an ISP is operably linked to control sequences permitting expression of the said nucleic acid sequence in a filamentous fungal host cell.

The term “operably linked” is defined herein as a configuration in which a control sequence is appropriately placed at a position relative to the ISP coding sequence such that the control sequence directs the production of an RNA or an mRNA and optionally of a polypeptide translated from said (m)RNA.

The term “control sequences” is defined herein to include all components, which are necessary or advantageous for the expression of mRNA and/or a polypeptide, either in vitro or in a host cell. Each control sequence may be native or foreign to the nucleic acid sequence encoding the polypeptide. Such control sequences include, but are not limited to, a leader, Shine-Delgarno sequence, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription terminator. Signal sequences used for optimizing expression are described in WO2010/121933. At a minimum, the control sequences include a promoter, and transcriptional and translational stop signals. Control sequences may be optimized to their specific purpose. Preferred optimized control sequences used in the present invention are those described in WO2006/077258, which is herein incorporated by reference.

One or more control sequences may be a control sequence which does not natively occur in Leucosporidium, for example a Leucosporidum from which the ISP was originally isolated.

The control sequences may be provided with linkers for the purpose of introducing specific restriction sites facilitating ligation of the control sequences with the coding region of the nucleic acid sequence encoding a polypeptide.

The control sequence may be an appropriate promoter sequence (promoter). The term “promoter” is defined herein as a DNA sequence that binds RNA polymerase and directs the polymerase to the correct downstream transcriptional start site of the nucleic acid sequence encoding an ISP. RNA polymerase effectively catalyzes the assembly of messenger RNA complementary to the appropriate DNA strand of a coding region. The term “promoter” will also be understood to include the 5′-non-coding region (between promoter and translation start) for translation after transcription into mRNA, cis-acting transcription control elements such as enhancers, and other nucleotide sequences capable of interacting with transcription factors.

A nucleic acid construct as used in the invention may be one wherein the control sequences comprise a promoter not natively associated with the nucleic acid encoding an ISP.

The promoter may be any appropriate promoter sequence suitable for a filamentous fungus host cell, which shows transcriptional activity, including mutant, truncated, and hybrid promoters, and may be obtained from polynucleotides encoding extra-cellular or intracellular polypeptides either homologous (native) or heterologous (foreign) to the filamentous fungal host cell. The promoter may be a constitutive or inducible promoter.

The promoter may be an inducible promoter. The promoter may be a carbohydrate inducible promoter. Carbohydrate inducible promoters that can be used are a starch-, cellulose-, hemicellulose (such as xylan- and/or xylose-inducible) promoters. Other inducible promoters are copper-, oleic acid-inducible promoters. Promoters suitable in filamentous fungi are promoters which may be selected from the group, which includes but is not limited to promoters obtained from the polynucleotides encoding A. oryzae TAKA amylase, Rhizomucor miehei aspartic proteinase, A. niger neutral alpha-amylase, A. niger acid stable alpha-amylase, A. niger or A. awamori glucoamylase (glaA), A. niger or A. awamori endoxylanase (xlnA) or beta-xylosidase (xlnD), T. reesei cellobiohydrolase I (CBHI), R. miehei lipase, A. oryzae alkaline protease, A. oryzae triose phosphate isomerase, A. nidulans acetamidase, Fusarium venenatum amyloglucosidase (WO 00/56900), Fusarium venenatum Dania (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase), and mutant, truncated, and hybrid promoters thereof. Other examples of promoters are the promoters described in WO2006/092396 and WO2005/100573, which are herein incorporated by reference. An even other example of the use of promoters is described in WO2008/098933 and PCT/EP2-13/062490. Promoters can also be constitutive promoters.

The control sequence may also be a suitable transcription terminator (terminator) sequence, a sequence recognized by a filamentous fungal cell to terminate transcription. The terminator sequence is operably linked to the 3′-terminus of the nucleic acid sequence encoding the polypeptide. Any terminator, which is functional in the cell, may be used in the present invention. The man skilled in the art knows which types of terminators can be used in the microbial host cell as described herein.

Preferred terminator sequences for filamentous fungal cells are obtained from any terminator sequence of a filamentous fungal gene, more preferably from Aspergillus genes, even more preferably from the gene A. oryzae TAKA amylase, the genes encoding A. niger glucoamylase (glaA), A. nidulans anthranilate synthase, A. niger alpha-glucosidase, trpC and/or Fusarium oxysporum trypsin-like protease.

The control sequence may also be an optimal translation initiation sequences (as described in Kozak, 1991, J. Biol. Chem. 266:19867-19870), or a 5′-untranslated sequence, a non-translated region of a mRNA which is important for translation by filamentous fungal host cell. The translation initiation sequence or 5′-untranslated sequence is operably linked to the 5′-terminus of the coding sequence encoding the polypeptide. Each control sequence may be native or foreign to the nucleic acid sequence encoding the polypeptide. Control sequences may be optimized to their specific purpose.

Suitable 5′-untranslated sequences may be those polynucleotides preceeding the fungal amyloglucosidase (AG) gene, A. oryzae TAKA amylase and Aspergillus triose phosphate isomerase genes and A. niger glucoamylase glaA, alpha-amylase, xylanase and phytase encoding genes.

The control sequence may also be a non-translated region of a mRNA which is important for translation by the filamentous fungus host cell.

A leader (or signal) sequence may be operably linked to the 5′-terminus of the nucleic acid sequence encoding the polypeptide. Any leader, which is functional in the cell, may be used in the present invention. Leader sequences may be those originating from the fungal amyloglucosidase (AG) gene (glaA-both 18 and 24 amino acid versions e. g. from Aspergillus), the α-factor gene (yeasts e.g. Saccharomyces and Kluyveromyces) or the α-amylase (amyE, amyQ and amyL) and alkaline protease aprE and neutral protease genes (Bacillus), or signal sequences as described in WO2010/121933.

Preferred leaders (or signal sequences) for filamentous fungal cells are obtained from the polynucleotides preceding A. oryzae TAKA amylase and A. niger glaA and phytase.

Other control sequences may be isolated from the Penicillium IPNS gene, or pcbC gene, the beta tubulin gene. All the control sequences cited in WO 01/21779 are herewith incorporated by reference.

The control sequence may also be a polyadenylation sequence, a sequence which is operably linked to the 3′-terminus of the nucleic acid sequence and which, when transcribed, is recognized by the microbial host cell (mutated or parent) as a signal to add polyadenosine residues to transcribed mRNA. Any polyadenylation sequence, which is functional in the cell, may be used in the present invention.

Preferred polyadenylation sequences for filamentous fungal cells are obtained from the polynucleotides encoding A. oryzae TAKA amylase, A. niger glucoamylase, A. nidulans anthranilate synthase, Fusarium oxysporum trypsin-like protease and A. niger alpha-glucosidase.

In order to facilitate expression, the nucleic acid sequence encoding the ISP may be a synthetic polynucleotide. Synthetic polynucleotides may be optimized in codon use, preferably according to the methods described in WO2006/077258 and/or PCT/EP2007/055943 (published as WO2008/000632), which are herein incorporated by reference. PCT/EP2007/055943 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the ISP and/or improved production of the encoded ISP. Codon pairs are defined as a set of two subsequent triplets (codons) in a coding sequence.

Accordingly, a nucleic acid construct as used in the present invention may be one wherein the nucleic acid encoding an ISP is codon pair optimized for expression in a filamentous fungal host cell.

In order to facilitate expression and/or translation of the ISP, the nucleic acid sequence encoding the ISP may be comprised in an expression vector such that the gene encoding the ISP is operably linked to the appropriate control sequences for expression and/or translation in vitro, or in the filamentous fungal host cell. That is to say, the invention describes an expression vector comprising a nucleic acid construct as used in the present invention.

The expression vector may be any vector (e.g., a plasmid or virus), which can be conveniently subjected to recombinant DNA procedures and can bring about the expression of the polynucleotide encoding the polypeptide. The choice of the vector will typically depend on the compatibility of the vector with the cell into which the vector is to be introduced. The vectors may be linear or closed circular plasmids. The vector may be an autonomously replicating vector, i. e., a vector, which exists as an extra-chromosomal entity, the replication of which is independent of chromosomal replication, e.g., a plasmid, an extra-chromosomal element, a mini-chromosome, or an artificial chromosome. An autonomously maintained cloning vector may comprise the AMA1-sequence (see e.g. Aleksenko and Clutterbuck (1997), Fungal Genet Biol. 21: 373-397).

Alternatively, the vector may be one which, when introduced into the filamentous fungal host cell, is integrated into the genome and replicated together with the chromosome(s) into which it has been integrated. The integrative cloning vector may integrate at random or at a predetermined target locus in the chromosomes of the host cell. Preferably, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the cell. Linearization is preferably performed such that at least one but preferably either end of the cloning vector is flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb. Preferably, the efficiency of targeted integration into the genome of the filamentous fungus host cell, i.e. integration in a predetermined target locus, is increased by augmented homologous recombination abilities of the host cell.

Preferably, the homologous flanking DNA sequences in the cloning vector, which are homologous to the target locus, are derived from a highly expressed locus meaning that they are derived from a gene, which is capable of high expression level in the host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1).

A number of preferred highly expressed fungal genes are given by way of example: the amylase, glucoamylase, alcohol dehydrogenase, xylanase, glyceraldehyde-phosphate dehydrogenase or cellobiohydrolase (cbh) genes from Aspergilli, Chrysosporium or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1, a Chrysosporium luckndowense cbh gene or a cbh gene from P. chrysogenum.

More than one copy of a nucleic acid construct may be inserted into a filamentous fungus host cell to increase production of the ISP (over-expression) encoded by the nucleic acid sequence comprised within the nucleic acid construct. This can be done, preferably by integrating into its genome copies of the DNA sequence, more preferably by targeting the integration of the DNA sequence at one of the highly expressed loci defined in the former paragraph. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase even more the number of copies of the DNA sequence to be over expressed the technique of gene conversion as described in WO98/46772 may be used.

The vector system may be a single vector or plasmid or two or more vectors or plasmids, which together contain the total DNA to be introduced into the genome of the host cell, or a transposon.

The vectors preferably contain one or more selectable markers, which permit easy selection of transformed cells. A selectable marker is a gene the product of which provides for biocide or viral resistance, resistance to heavy metals, prototrophy to auxotrophs, and the like. The selectable marker may be introduced into the cell on the expression vector as the expression cassette or may be introduced on a separate expression vector.

A selectable marker for use in a filamentous fungal cell may be selected from the group including, but not limited to, amdS (acetamidase), argB (ornithine carbamoyltransferase), bar (phosphinothricinacetyttransferase), bleA (phleomycin binding), hygB (hygromycinphosphotransferase), niaD (nitrate reductase), pyrG (orotidine-5′-phosphate decarboxylase), sC (sulfate adenyttransferase), NAT or NTC (Nourseothricin) and trpC (anthranilate synthase), as well as equivalents from other species. Preferred for use in an Aspergillus and Penicillium cell are the amdS (see for example EP 635574 B1, EP0758020A2, EP1799821A2, WO 97/06261 A2) and pyrG genes of A. nidulans or A. oryzae and the bar gene of Streptomyces hygroscopicus. More preferably an amdS gene is used, even more preferably an amdS gene from A. nidclans or A. niger. A most preferred selectable marker gene is the A. nidulans amdS coding sequence fused to the A. nidulans gpdA promoter (see EP 635574 B1). Other preferred AmdS markers are those described in WO2006/040358. AmdS genes from other filamentous fungi may also be used (WO 97/06261).

Preferably, any selection marker is deleted from the transformed filamentous fungus host cell after introduction of the expression construct so as to obtain transformed host cells capable of producing the ISP which are free of selection marker genes.

The procedures used to ligate the elements described above to construct the expression vectors are well known to one skilled in the art (see, e.g. Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995).

Furthermore, standard molecular cloning techniques such as DNA isolation, gel electrophoresis, enzymatic restriction modifications of nucleic acids, Southern analyses, transformation of cells, etc., are known to the skilled person and are for example described by Sambrook et al. (1989) “Molecular Cloning: a laboratory manual”, Cold Spring Harbor Laboratories, Cold Spring Harbor, N.Y. and Innis et al. (1990) “PCR protocols, a guide to methods and applications” Academic Press, San Diego.

A nucleic acid suitable for use in the invention may be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis.

Preferably, the efficiency of targeted integration into the genome of the host cell, i.e. integration in a predetermined target locus, is increased by augmented homologous recombination abilities of the host cell. Such phenotype of the cell preferably involves a deficient hdfA or hdfB as described in WO2005/095624. WO2005/095624 discloses a preferred method to obtain a filamentous fungal cell comprising increased efficiency of targeted integration

The invention thus describes a filamentous fungal host cell which comprises a nucleic acid construct or an expression vector as described herein.

The filamentous fungal host cell may be a cell of any filamentous form of the subdivision Eumycota and Oomycota (as defined by Hawksworth at al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic.

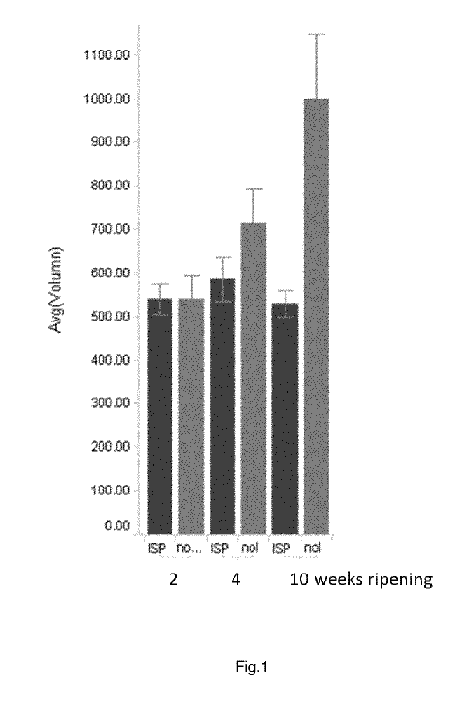

The filamentous fungal host cell may be a cell of any filamentous form of the taxon Trichocomaceae (as defined by Houbraken and Samson in Studies in Mycology 70: 1-51. 2011). In another preferred embodiment, the filamentous fungal host cell may be a cell of any filamentous form of any of the three families Aspergillaceae, Thermoascaceae and Trichocomaceae, which are accommodated in the taxon Trichocomaceae. Suitable filamentous fungal host cells may be those in Clade 2: Aspergillus as described in FIG. 1 of Houbraken and Samson, 2011 (supra).

Suitable filamentous fungal host cells suitable for use in the invention include, but are not limited to, cells of Acremonium, Agaricus, Aspergillus, Aureobasidium, Chrysosporium, Coprinus, Cryptococcus, Filbasidium, Fusarium, Humicola, Magnaporthe, Mucor, Myceliophthora, Neocallimastix, Neurospora, Paecilomyces, Penicilium, Piromyces, Panerochaete, Pleurotus, Schizophyllum, Talaromyces, Rasamsonia, Thermoascus, Thielavia, Tolypoclahdum, and Trichoderma.

Preferred filamentous fungal cells belong to a species of an Acremonium, Aspergillus, Chrysosporium, Mycelophthora, Penicilium, Talaromyces, Rasamsonia, Thielavia, Fusarium or Trichoderma genus, and most preferably a species of Aspergillus niger, Acremonium alabamense, Aspergillus awamori, Aspergillus foetidus, Aspergillus sojae, Aspergillus fumigatus, Talaromyces emersonii, Rasamsonia emersonii, Aspergillus oryzae, Chrysosporium lucknowense, Fusarium oxysporum, Fusarium venenatum, Myceliophthora thermophila, Trichoderma reesei, Thielavia terrestris or Penicillium chrysogenum. A more preferred host cell belongs to the genus Aspergillus, more preferably the host cell belongs to the species Aspergillus niger. When the host cell is an Aspergillus niger host cell, the host cell preferably is CBS 513.88, CBS124.903 or a derivative thereof. Another preferred host cell belongs to the species Aspergillus oryzae. I.e. preferably, the ISP used in a method or food product of the invention, is produced in an Aspergillus host cell and more preferably in Aspergillus niger or Aspergillus oryzae.

Several strains of filamentous fungi are readily accessible to the public in a number of culture collections, such as the American Type Culture Collection (ATCC), Deutsche Sammlung von Mikroorganismen und Zelkuilturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Useful strains in the context of the present invention may be Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255 (ATCC28089), Penicillium citrinum ATCC 38065, Penicilium chrysogenum P2, Rasamsonia emersonii ATCC16479, CBS393.64, IFO31232, IM1116815, Thietavia terrestris NRRL8126, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceiophthora thermophia C1, Garg 27K, VKM-F 3500 D, Chrysosporium fucknowense C1, Garg 27K, VKM-F 3500 D, ATCC44006 and derivatives thereof.

Preferred filamentous fungus host cells such as A. niger host cells, for example possibly contain one, more or all of the following modifications: deficient in a non-ribosomal peptide synthase preferably deficient in a non-ribosomal peptide synthase npsE (see WO2012/001169), deficient in pepA, deficient in glucoamylase (glaA), deficient in acid stable alpha-amylase (amyA), deficient in neutral alpha-amylase (amyBI and amyBII), deficient in oxalic acid hydrolase (oahA), deficient in one or more toxins, preferably ochratoxin and/or fumonisin, deficient in prtT, deficient in hdfA, comprises a SEC 61 modification being a S376W mutation in which Serine 376 is replaced by Tryptophan and/or comprises an adapted amplicon as defined in WO2005/123763 and/or WO2011/009700. These and other possible host modifications are also described in WO2012/001169, WO2011/009700, WO2007/062936, WO2006/040312 or WO2004/070022, WO2013/135729, WO2014/013074, WO2014/013073.

Those skilled in the art know how to transform cells with the one or more nucleic acid construct or expression vector.

Transformation of the filamentous fungal host cell may be conducted by any suitable known methods, including e.g. electroporation methods, particle bombardment or microprojectile bombardment, protoplast methods and Agrobacterium mediated transformation (AMT). Procedures for transformation are described by J. R. S. Fincham, Transformation in fungi. 1989, Microbiological reviews. 53, 148-170.

Transformation may involve a process consisting of protoplast formation, transformation of the protoplasts, and regeneration of the cell wall in a manner known per se. Suitable procedures for transformation of Aspergillus cells are described in EP 238 023 and Yelton et al., 1984, Proceedings of the National Academy of Sciences USA 81:1470-1474. Suitable procedures for transformation of Aspergillus and other filamentous fungal host cells using Agrobacterium tumefaciens are described in e.g. De Groot et al., Agrobacterium tumefaciens-mediated transformation of filamentous fungi. Nat Biotechnol. 1998, 16:839-842. Erratum in: Nat Biotechnol 1998 16:1074. A suitable method of transforming Fusarium species is described by Malardier et al., 1989, Gene 78:147156 or in WO 96/00787. Other methods can be applied such as a method using biolistic transformation as described in: Christiansen et al., Biolstic transformation of the obligate plant pathogenic fungus, Erysiphe graminis f.sp. hordei. 1995, Curr Genet 29:100-102.

As described above the ISP used in any method of the invention or the ISP present in a food product of the invention is preferably produced by using a nucleic acid construct which comprises:
a nucleic acid sequence encoding an ice structuring protein (ISP) comprising the sequence set out in SEQ ID NO: 1 or a sequence at least 80% identical thereto or comprising the sequence set out in amino acids 21 to 261 of SEQ ID NO: 1 or a sequence at least 80% identical thereto; and, linked operably thereto,
control sequences permitting expression of the nucleic acid sequence in a filamentous fungal host cell.

Alternatively, the ISP used in any method of the invention or the ISP present in a food product of the invention is produced by a method for the production of an ice structuring protein (ISP), which method comprises:
providing a filamentous fungal host cell which comprises a nucleic acid sequence encoding an ISP comprising the sequence set out in SEQ ID NO: 1 or a sequence at least 80% identical thereto or comprising the sequence set out in amino acids 21 to 261 of SEQ ID NO: 1 or a sequence at least 80% identical thereto,
wherein the said nucleic acid sequence is operably linked to control sequences permitting expression of the nucleic acid sequence in the filamentous fungal host cell;
cultivating the filamentous fungal host cell under conditions suitable for production of the ice structuring protein; and, optionally
recovering the ice structuring protein.

Typically, the ISP is secreted from the host cell, for example during the cultivation step.

In step a. a mutant microbial host cell may be a filamentous fungus host cell as described herein.

In step b. the filamentous fungus host cell of step a. is cultured under conditions conducive to the expression of the ISP. The mutant microbial cells are cultivated in a nutrient medium suitable for production of the ISP using methods known in the art. For example, the cells may be cultivated by shake flask cultivation, small-scale or large-scale fermentation (including continuous, batch, fed-batch, or solid state fermentations) in laboratory or industrial fermentors performed in a suitable medium and under conditions allowing the ISP to be produced and/or isolated. The cultivation takes place in a suitable nutrient medium comprising carbon and nitrogen sources and inorganic salts, using procedures known in the art (see, e. g., Bennett, J. W. and LaSure, L., eds., More Gene Manipulations in Fungi, Academic Press, C A, 1991). Suitable media are available from commercial suppliers or may be prepared using published compositions (e. g., in catalogues of the American Type Culture Collection).

If the ISP is secreted into the nutrient medium, the ISP can be isolated directly from the medium. If the ISP is not secreted, it can be isolated from cell lysates.

In step c., the ISP may be optionally isolated. The ISP may be isolated by methods known in the art. For example, the ISP may be isolated from the nutrient medium by conventional procedures including, but not limited to, centrifugation, filtration, extraction, spray drying, evaporation, or precipitation. The isolated ISP may then be further purified by a variety of procedures known in the art including, but not limited to, chromatography (e. g., ion exchange, affinity, hydrophobic, chromatofocusing, and size exclusion), electrophoretic procedures (e.g., preparative isoelectric focusing), differential solubility (e. g., ammonium sulfate precipitation), or extraction (see, e.g., Protein Purification, J.-C. Janson and Lars Ryden, editors, VCH Publishers, New York, 1989). In some applications the ISP may be used without substantial isolation from the culture broth; separation of the culture medium from the biomass may be adequate.

Preferably, productivity of the ISP may be at least 1 g/L, at least 2 g/L, at least 5 g/L, such as 10 g/L or higher.

Alternatively, the ISP used in any method of the invention or the ISP present in a food product of the invention comprises the sequence set out in SEQ ID NO: 1 or a sequence at least 80% identical thereto or comprising the sequence set out in amino acids 21 to 261 of SEQ ID NO: 1 or a sequence at least 80% identical thereto, wherein:

at least one amino acid is a modified amino acid, for example comprising a pyroglutamate modification at its N-terminus;

at least one amino acid is O-mannosylated, for example comprising one, two, three, four or more O-mannosylations;

the protein has a glycosylation pattern other than 2GlcNac and 2 hexose units, for example 2GlcNac and three, four, five, six, seven, eight, nine, ten or more hexose units; or

the protein lacks WQKRSNARQWL, VQKRSNARQWL or KRSNARQWL at the C-terminus.

That is to say, the protein may have a C-terminal truncation of one, two, three, four, five, six, seven, eight, nine, ten, eleven or twelve or more amino acids with reference to the protein set out in SEQ ID NO: 1.

In such an ice structuring protein, the modified amino acid may be a pyroglutamate, optionally present at the N-terminus of the protein (i.e. at an amino acid corresponding to amino acid 21 in SEQ ID NO: 1).

An ice structuring protein used in a method of food product of the invention may comprise 2 N-acetylglucosamine (GlcNAc) and 10 hexose (Hex) units. An ice structuring protein used in a method of food product may be O-mannosylated at a position corresponding to S80 and/or T84 with reference to SEQ_ID NO:1. The most abundant form of AFP19 contains one O-mannosyl group.

An ISP used in a method or food product as described herein may comprise the amino acid sequence set out in SEQ ID NO: 1 or a sequence at least 85%, at least 90%, at least 95%, at least 98% or at least 99% identical thereto or comprising the sequence set out in amino acids 21 to 261 of SEQ ID NO: 1 or a sequence at least 85%, at least 90%, at least 95%, at least 98% or at least 99% identical thereto.

The invention, amongst others, relates to a method for preparing a food product which food product is frozen and completely thawed before consumption (i.e. a method for preparing a frozen food product which is completely thawed before consumption), which method comprises incorporating an ice structuring protein (ISP) in said food product and freezing the prepared food product (and optionally storing the prepared food product at frozen conditions). Accordingly, the invention also provides a (frozen) food composition comprising an ISP, for example as obtainable by a method of the invention.

Preferably, the food product is a water-continuous dispersion, such as cream, yoghurt, cheese, mayonnaise or a dressing. This means that a separate, water-immiscible phase is dispersed in smaller entities in a continuous aqueous phase. The dispersed phase can consist of oils, fats, polymeric particles—consisting of synthetic polymers or biopolymers such as proteins, an organic phase, containing for instance monomers that later polymerize, surface active components (in detergents, shampoos and other personal products, certain fire extinguishers), or solid particles. The polymers or particles can also form a network in the continuous aqueous phase, such as happens in for instance yoghurt or starch dispersions (e.g. custard). Without being bound by it, the inventors of the present invention hypothesize that upon freezing ice crystals force the dispersed phase particles onto each other, leading to often irreversible aggregation and/or coalescence. After thawing the material will not return in its original finely dispersed state. Consequently large product defects occur such as phase separation, coalescence, formation of cracks in the polymeric network, textural breakdown, clearly observable macroscopically. Temperature cycling will even amplify this destructive effect. Controlling ice crystal size keeping ice crystals small during the frozen state will therefore keep the dispersed phase intact and in its original state and thereby maintain product stability.

More preferably, said food product which is frozen and thawed before consumption is a dairy food product such as cream or cheese. Even more preferably, the food product is completely thawed before consumption. Food products such as frozen confectionery products, such as ice-cream, frozen yoghurt, frozen desserts, sherbet, sorbet, ice milk, frozen custard, water-ices, granitas and frozen fruit purees, soft serve, frappé, slush, smoothies, shave ice, snow cones, semifreddo, milk shakes or gelato are not within the scope of the present invention.

The term “cream” is used herein to refer to a dairy product that is composed of the higher-butterfat layer skimmed from the top of milk before homogenization. In un-homogenized milk, the fat, which is less dense, will eventually rise to the top. In the industrial production of cream, this process is accelerated by using centrifuges called “separators”. In many countries, cream is sold in several grades depending on the total butterfat content. Cream can be dried to a powder for shipment to distant markets. The cream as used in a method of the invention may also be a reconstituted cream, i.e. the cream as used herein may be (fresh) cream or reconstituted cream. Fat levels in cream depend on the application and typically cream will at least contain 10% lipids (w/w on wet base). Cream used to make butter or butter oil usually contains at least 30% of lipids.

A typical, non-limiting, cheese making process involves the next steps: standarization of milk, pasteurization or heat treatment of the milk, cooling of the milk, inoculation with starter cultures and optional non-starter adjunct cultures, addition of coagulant, formation of curd, cutting of curd, draining of whey, optional salting, storage, aging and packaging of the resulting cheese. Preferably, the ISP is added together with or just after the addition of the starter culture, adjunct culture and coagulant. Hundreds of types of cheese from various countries exist and the invention is applicable to most of them. Preferably, the cheese is a semi-hard cheese such as Gouda cheese, Cheddar cheese or Leerdammer cheese or a pasta filata-type cheese like Mozzarella.

The level of ISP may be from 0.00001 to 0.5% by weight based on the final composition. The skilled person is capable to determine an effective amount of ISP.

The invention also provides use of an ice structuring protein for preparing a food product which is frozen and completely thawed before consumption (i.e. a frozen food product which is completely thawed before consumption). More preferably, the invention also provides use of an ice structuring protein for preparing frozen/thawed cream or cheese. In more detail, the invention provides use of an ice structuring protein which comprises the sequence set out in SEQ ID NO: 1 or a sequence at least 80% identical thereto or comprising the sequence set out in amino acids 21 to 261 of SEQ ID NO: 1 or a sequence at least 80% identical thereto, wherein:
at least one amino acid is a modified amino acid;
at least one amino acid is O-mannosylated;
the protein has a glycosylation pattern other than 2GlcNac and 2 hexose units; or
the protein lacks WQKRSNARQWL, VQKRSNARQWL or KRSNARQWL at the C-terminus
   for preparing a frozen food product which frozen food product is completely thawed before consumption. Preferably, said frozen food product is a frozen dairy product and more preferably said frozen food product is frozen cheese or frozen cream.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The present invention is further illustrated by the following Examples:

### EXAMPLES

### Example 1: Cloning and Expression of the AFP19 Gene

The protein sequence of the ISP of Leucosporidium (AFP19) was deduced from the published gene sequence and is shown in SEQ ID NO: 1. This sequence consists of a signal sequence of 20 amino acids for efficient secretion in Leucosporidium, and a deduced mature protein sequence of 241 amino acids.

A codon-adapted DNA sequence for expression of this protein in Aspergillus niger was designed containing additional restriction sites for subcloning in an Aspergillus expression vector. Codon adaptation was performed as described in WO2008/000632. The DNA sequence of the gene encoding the ISP protein of SEQ ID NO:1 is shown in SEQ ID NO: 2.

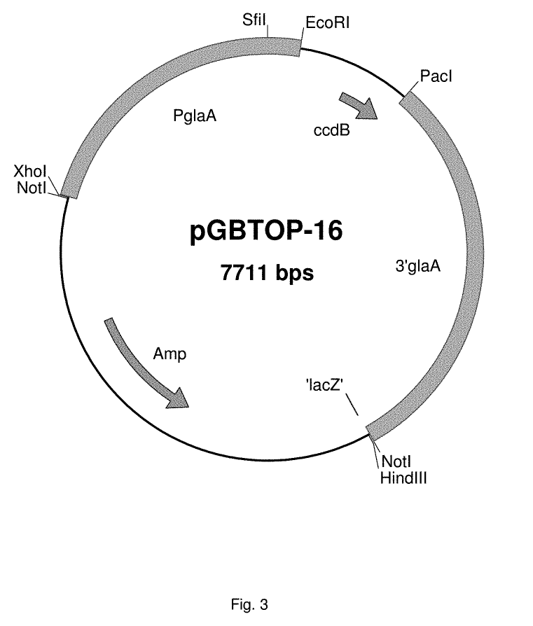

The translational initiation sequence of the glucoamylase glaA promoter has been modified into 5′-CACCGTCAAA ATG-3′ and an optimal translational termination sequence 5′-TAAA-3′ was used in the generation of the expression construct (as also detailed in WO2006/077258). A DNA fragment (SEQ ID NO: 3), containing a.o. part of the glucoamylase promoter and the ISP encoding gene, was synthesized completely, purified and digested with EcoRI and PacI. The pGBTOP-16 vector (FIG. 3) was linearized by EcoRI/PacI digestion and the linearized vector fragment was subsequently purified by gel-extraction. The DNA fragment was cloned into the pGBTOP-16 vector and the resulting vector was named pGBTOPAFP-19. Subsequently, A. niger GBA 306 was transformed with this pGBTOPAFP-19 vector, in a co-transformation protocol with pGBAAS-4, with strain and methods as described in WO 2011/009700 and references therein, and selected on acetamide containing media and colony purified according to standard procedures. A. niger GBA306 is ultimately derived from CBS124.903 (deposited at the Centraabureau voor Schimmelcultures, Utrecht, the Netherlands) Transformation and selection was performed as described in WO 98/46772 and WO 99/32617. Strains containing the AFP19 gene were selected via PCR with primers specific for the AFP19 gene to verify presence of the pGBTOPAFP-19 expression cassette. A single transformant was selected, named AFP19-3, and further replica-plated to obtain a single strain inoculum.

### Example 2: Fermentation and Purification of AFP19 in Aspergillus niger

Fresh A. niger AFP19-3 spores were prepared. 4 shake flasks with 100 ml Fermentation medium 1 (10% w/v Corn Steep Solids, 1% w/v glucose.H₂O, 0.1% w/v NaH₂PO₄.H₂O, 0.05% w/v MgSO₄.7H₂O, 0.025% w/v Basildon, pH 5.8) in 500 ml shake flasks with baffle were inoculated with 10⁷ spores. These pre-cultures were incubated at 34° C. and 170 rpm for 16-24 hours. From the pre-cultures, 50 ml was used for inoculation of 4 shake flasks with 1 liter Fermentation medium 2 (15% w/v maltose, 6% w/v bacto-soytone, 1.5% w/v (NH₄)₂SO₄, 0.1% w/v NaH₂PO₄.H₂O, 0.1% w/v MgSO₄.7H₂O, 0.1% w/v L-arginine, 8%. w/v Tween-80, 2%. w/v Basildon, 2% w/v MES pH 5.1) in a 5 liter shake flask size and shaken at 34° C. and 170 rpm. After four days of cultivation, the cells were killed off by adding 3.5 g/l of sodium benzoate and keeping at 30° C. for six hours. Subsequently, 10 g/l CaCl2 and 45 g/l Perlite C25 was added to the culture broth. Filtration was carried out in one step using filter cloth and filters DE60/EKS P and K250 (Pall). The filter cake remaining at the filter was washed with 1.1 l of sterile milliQ water. Subsequent sterile filtration was carried out using 0.22 m GP Express PLUS Membrane (Millipore). Ultrafiltration was carried out on the Pellicon 2 “mini” ultrafiltration system with the cassette Biomax5k (Millipore) and washed with 50 mM Na-acetate, pH 5.6. The protein composition of purified samples at all steps was controlled by 4-12% SDS-PAGE and found to be >90% pure AFP19. The protein concentration was controlled at all steps by measuring A260/A280. The concentration was determined according to the formula c(mg/ml)=(1.55*A280)−(0.76*A260). Finally a 150 ml sample was obtained with a protein concentration 30.8 mg/ml, indicating that the productivity of AFP19 was >1 g/kg fermentation broth. The final preparation was freeze dried and stored at −20° C. until use.

### Example 3: Expression and Fermentation of the AFP19 Gene in Aspergillus oryzae

Aspergillus oryzae strain CBS205.89 (deposited at the Centraabureau voor Schimmelcultures, Utrecht, the Netherlands and publically available) was used as host and circular vectors pGBTOPAFP-19 vector and pGBAAS-4, described in Example 1, were used in a co-transformation protocol. Transformation was performed as described in WO 98/46772 and WO 99/32617, and selection was on acetamide containing media with 20 mM cesium chloride added. Colonies were purified according to standard procedures. Strains containing the AFP19 gene were selected via PCR with primers specific for the AFP19 gene to verify presence of the pGBTOPAFP-19 expression cassette. A single transformant was selected, named AFPao-7, and further replica-plated to obtain a single strain inoculum. As control a strain of Aspergillus oryzae was transformed with plasmid pGBTOP-16 instead of pGBTOAFP-19, and a single strain inocumum was obtained by replica plating. Fresh A. oryzae AFPao-7 spores were prepared and cultured as described in example 2. Cultivation was performed for 72 hours at 30° C. and 170 rpm in shake flask. Supernatant was harvested by centrifugation and stored at −20° C. until further analysis.

The concentration of AFP19 protein produced by Aspergillus oryzae AFPao-7 was determined using SDS-PAGE analysis of the supernatant and comparison with a serial dilution of the relatively pure AFP19 from Aspergillus niger from Example 2. Cultivation supernatant of the control strain lacking the AFP19 gene was analysed in parallel on the same gel. After staining the gel with Coomassie Brilliant Blue, the staining was quantified and compared to the serial dilution of AFP19 from A. niger. The amount of AFP19 produced by Aspergillus oryzae was estimated to be 0.4 g/l, clearly more than the 61.2 mg/i of LelBP produced in shake flask in Pichia pastoris (Park et al. 2012, supra). This amount was confirmed by measuring the total protein concentration in the supernatant using Bradford protein stain after deducing the staining intensity of the supernatant of the control strain.

### Example 4: Analysis of AFP19 from Aspergillus niger

AFP19 as isolated in Example 2 from Aspergillus niger was further characterized using LC-MS/MS. For this 1 mg AFP19 protein as isolated in Example 2 was diluted until 100 μg/ml in 100 mM NH4HCO3. For deglycosylation, 100 μl of this solution was heated for 10 min at 90° C. 15 μl PNGase F (Sigma, 1 U/μl) was added and then incubated for 4 hours in a thermomixer at 1000 rpm and 37° C. 1% formic acid was added to the samples before measuring. As a control untreated AFP19 protein was analyzed.

For the LC-MS/MS analysis the samples were analyzed on the Acquity I-class—Synapt G2-S (Waters), with the following parameters: Column: Waters Acquity UPLC BEH300 C4 1.71 μm 300 Å pore size 2.1×50 mm column. Column temperature: 75° C. Injection volume: 5 μl. Mobile phase A: Formic Acid 0.1% in Water. Mobile phase B: Formic Acid 0.1% in Acetonitrile. A gradient was applied to the column by varying phase A and B in order to separate different forms of the AFP19 protein.

The MS detector settings were: Acquisition mass range was 500-3500 m/z, Scan time 1 sec, Positive ESI, TOF MS Resolution mode, with data correction with Leu-Enk applied on the fly during the run. Data spectral deconvolution, charge state stripping, was performed with a Waters MassLynx MaxEnt1-software tool: Output mass−resolution=1 Da/channel. Damage model: Gaussian (FWHH=0.750 Da; minim intensity ratios=33% left and right). Iterate to converge.

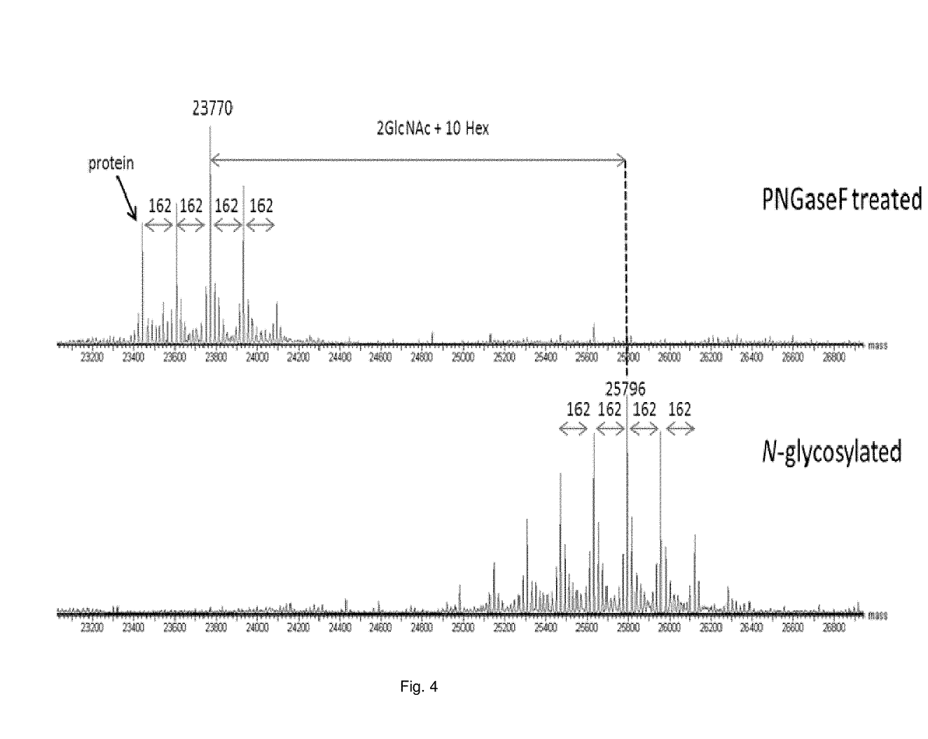

The mass of AFP19 was determined before and after enzymatic deglycosylation using this technique and results are depicted in FIG. 4.

Several conclusions can be drawn from these results:

The size of the smallest form of AFP19 after PNGase F treatment is 23446 Da. This size is smaller than the calculated molecular weight based on the protein sequence depicted in SEQ ID NO:1 assuming the removal of the pre-sequence after residue 20. This indicates that the AFP19 produced in A. niger is missing part of the protein sequence. Since the size does not fit exactly the size of any amino acid deletion, we cannot exclude that other modifications may have occurred during production of AFP19 in A. niger. E.g. it is possible that AFP19 contains a pyroglutamate modification at its N-terminus.

AFP19 is heterogenous is size, even after PNGase F treatment. Four peaks with an increment of 162 Da (the size of one hexose unit) are found after PNGase F treatment and the most abundant form being 23770 Da. This indicates that AFP19 produced in A. niger contains additional glycosylation, that is not removed by PNGase F. AFP19 may be O-mannosylated up to 4 mannosyl residues per molecule of AFP19, with the most abundant form having 2 mannosyl groups. No O-mannosylation has been detected with the different forms of LelBP studied in the prior art (Lee et al, 2012, Journal of Biological Chemistry 287, 11460-11468; Lee et al, 2013 supra)

The size difference between the most abundant N-glycosylated form and most abundant deglycosylated form is 2026 Da, indicating that the main N-glycosylated form contains 2 N-acetylglucosamine (GlcNac) and 10 hexose (Hex) units, besides the 2 O-mannoses. The minimum size difference between N-glycosylated AFP19 and PNGase F treated AFP19 is 892 Da, representative for 2 GlcNac and 3 Hex units. This result indicates that all AFP19 forms produced in A. niger are differently N-glycosylated (and O-mannosylated) than LelBP produced in P. pastoris which was shown to contain only 2 GlcNac and 2 Hex (Bma and Man) units (Lee et al, 2012).

Using this method it became apparent that the molecular weight of the most abundant form of AFP19 produced in Aspergillus niger is 25796 Da. This size is different from the size measured by MALDI-TOF for both the native LelBP and both non-glycosylated and N-glycosylated forms produced in E. coli or P. pastoris (Lee et al., 2013).

These results indicate that AFP19 is clearly different from the different forms of LelBP studied in the prior art.

### Example 5: Analysis of AFP19 from Aspergillus niger and Aspergillus oryzae

AFP19 as isolated in Example 2 (Aspergillus niger) and Example 3 (Aspergillus oryzae) were further characterized using LC-MS/MS. For this 1 mg AFP19 protein was diluted until 100 μg/ml in 100 mM NH₄HCO₃. 100 μl of this solution was heated for 10 min at 90° C. AFP19 was analyzed as such, after deglycosylation with PNGase F, after digestion with LysC and after digestion with AspN. For deglycosylation, 15 μl PNGase F (Sigma, 1 U/μl) was added and then incubated for 16 hours in a thermomixer at 1000 rpm and 37° C. 1% formic acid was added to the samples before measuring. As a control untreated AFP19 protein was analyzed.

For the LC-MS/MS analysis the samples were analyzed on the Acquity I-class—Synapt G2-S (Waters), with the following parameters: Column: Acquity UPLC BEH300 C4 1.71 μm 300 Å pore size 2.1×50 mm column (Waters). Column temperature: 75° C. Injection volume: 1 μl. Mobile phase A: Formic Acid 0.1% in Water. Mobile phase B: Formic Acid 0.1% in Acetonitrile. A gradient was applied to the column by varying phase A and B in order to separate different forms of the AFP19 protein.

The MS detector settings were: Acquisition mass range was 500-3500 m/z, Scan time 1 sec, Positive ESI, TOF MS Resolution mode, with data correction with Leu-Enk applied on the fly during the run. Data spectral deconvolution, charge state stripping, was performed with a Waters MassLynx MaxEnt1-software tool: Output mass−resolution=1 Da/channel. Damage model: Gaussian (FWHH=0.750 Da; minim intensity ratios=33% left and right). Iterate to converge. Data spectral deconvolution for ETD, was performed with MassLynx MaxEnt3-software tool for max of 7 charges: no. of ensemble members: 2 and iteration per ensemble member: 50.

For digestion of AFP 19, Lys C and Asp N were used in parallel. 20 μl (2 μg) Lys C or Asp N was added to 100 μl (100 μg) and 400 μl (100 μg) AFP19, respectively. Digestion was performed by incubation at 37° C. overnight. Samples were diluted twice in MilliQ water and samples were acidified to 1% formic acid prior to LC-MS/MS analysis. LC-MS/MS analysis of the digested AFP19 samples were performed on the Ultimate RS 3000 Orbitrap Fusion (Thermo Fisher) with the following parameters: Column: Zorbax XDB-C18 1.8 μm 2.1×50 mm; narrow-bore guard column 2.1×12.5 mm 5-micron, Phoroshell 300SB-C3 (Agilent). Column temperature: 50° C. Injection volume: 25 μl. Mobile phase A: Formic Acid 0.1% in Water. Mobile phase B: Formic Acid 0.1% in Acetonitrile. A gradient was applied to the column by varying phase A and B.

The data were searched against the AFP19 sequence (FDR 0.1%). Database searching was performed on the Proteome Discoverer 1.4.1.14.

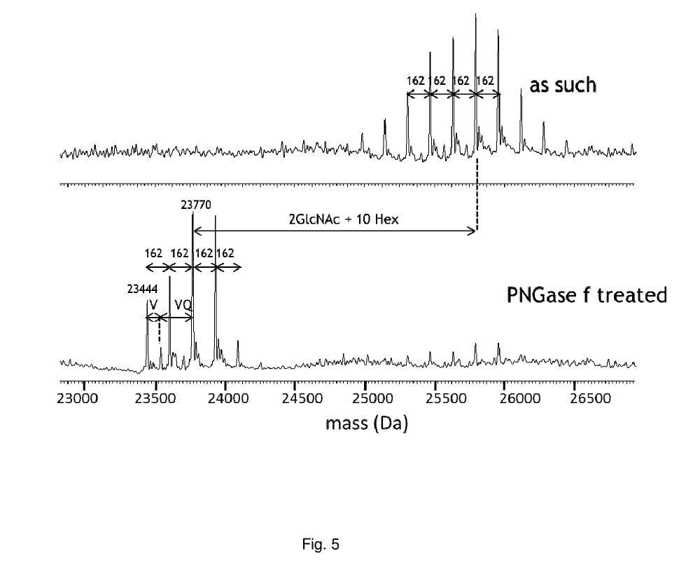

The deconvoluted mass spectra of intact AFP19 before and after PNGase F deglycosylation are shown In FIG. 5.

These data showed that the AFP19 expressed in A. niger has a pyroglutamate at the amino-terminus. This observation was confirmed by Electron Transfer Dissociation (ETD) on the intact enzyme as well as by identification of the N-terminal peptide after LysC digestion of AFP19. Pyroglutamate can play an important role in enzyme stability and has not been described in previous literature on LelBP (Lee et al, 2012 supra; Lee et al, 2013 supra).

Comparing the LC-MS data on intact AFP19 before and after deglycosylation showed that the main N-glycosylated form contains 2 N-acetyiglucosamine (GlcNAc) and 10 hexose (Hex) units as indicated in FIG. 3. The deglycosylated AFP19 showed mass increments of 162 Da, indicating that the enzyme is also O-mannosylated, next to the N-glycosylation. These findings were confirmed on the AspN digest of AFP19, where O-mannosylation was identified on position S80 and T84 in SEQ_ID NO:1. The most abundant form of AFP19 contains one O-mannosyl group. Besides the amino-terminal pyroglutamate, also O-mannosylation may have a positive effect on enzyme stability. Again, these findings have not been described in previous literature on AFP (Lee et al, 2012 supra; Lee et al, 2013 supra). The observations on both the N-glycosylation and the O-mannosylation showed that the glycosylation pattern of AFP19 described here is distinctly different from the native LelBP or LelBP produced in Pichia pastoris (Lee et al, 2012 supra).

The LC-MS analysis on intact AFP19 before and after deglycosylation further showed that the enzyme expressed in A. niger has a truncation at the C-terminus. The observed masses 23444 Da, 23543 Da and 23770 Da, respectively correspond to AFP19 with pyroglutamate at the N-terminus and lacking WQKRSNARQWL, VQKRSNARQWL and KRSNARQWL at the C-terminus.

The above mentioned experiments were also performed on AFP19 expressed in Aspergillus oryzae (sample as produced in Example 3). These data confirmed that expression in A. oryzae also results in AFP with pyroglutamate at the N-terminus and the N-glycosylation and 0-mannosylation patterns were highly similar to that of AFP19 expressed in A. niger. Furthermore neither the mass of native LelBP (25565 Da), from E. coli, nor the mass of glycosylated LelBP (26198 Da) and non-glycosylated (25150 Da) LelBP from P. pastoris reported before (Lee et al., 2013) were detected in our samples.

### Example 6: Ice Re-Crystallization Inhibition with AFP from Aspergillus niger

The RI endpoint for type III AFP from ocean pout in 30% sucrose was reported to be >700 nM by two different authors (Smallwood et al., 1999, supra, Tomczak et al., 2003, supra). The RI endpoint is the concentration below which RI activity was no longer detected. Since the RI endpoint is an important parameter for determination of the effectiveness of an ISP in the inhibition of ice-recrystallization, we decided to determine the RI endpoint in 30% sucrose for AFP19 using the modified splat assay essentially as described in Tomczak et al., 2003, supra.

A 30% (w/v) sucrose solution was supplemented with 100 mg/l whey protein isolate (WPI—Mullins whey) as control or 4000, 80 and 20 nM of AFP19 from Aspergillus niger prepared according to Example 2. For this experiment 23 mg AFP19 was dissolved in 4.6 ml distilled water and from this solution 50-, 2500-, 10000-fold dilutions were made in the 30% sucrose solution. 10 microliter of these solutions was used for the preparation of the microscopic samples and mounted onto a Zeiss Axiophot microscope equipped with a cooling stage. Imaging was performed using a CCD camera equipped on the microscope. Magnification was 6.3 fold and crystal formation in the samples was followed in time.

First the samples were cooled at a rate of 90° C. per second until −60° C. After this the samples were warmed at the same rate until −6° C. and ice crystal formation was followed in time. After 0, 3, 9 and 60 minutes pictures were taken and used for the measurement of the average ice crystal size using Linksys32 software (Linkam Scientific Instruments). Results of this experiment are shown in Table 1 which shows clearly that AFP19 inhibits ice recrystallization. No growth of ice crystals in time could be detected even when the concentration of AFP19 is only 20 nM. The control sample with WPI shows however a clear increase in the crystal size in time.

These results show that the RI endpoint in 30% sucrose for AFP19 is clearly lower than 20 nM. This value is much lower than the RI endpoint reported for type III AFP from ocean pout (Tomczak et al, 2003 supra: 780 nM). Thus, AFP19 is much more effective than the current industry standard type III HPLC12 AFP in inhibition of ice-recrystallization.

TABLE 1
Minimum and maximum size [micrometer] of ice crystals in time

| | T = 3 min | T = 9 min | T = 60 min |
|---|---|---|---|
| control | 8-10 | 15-93 | 24-185 |
| 4000 nM AFP19 | 5-11 | 6-9 | 4-13 |
| 80 nM AFP19 | 8-25 | 9-29 | 10-30 |
| 20 nM AFP19 | 8-27 | 7-29 | 7-33 |

### Example 7: Freezing and Thawing of Semi-Hard Cheese

Gouda Jong, Gouda Extra Belegen and Leerdammer Original cheese was purchased in a local supermarket. The cheeses were cut in cubes of approximately 2-3 cm and each cube was packed in a plastic container. Part of the samples were frozen and stored at −20° C. for 1 week. As a control, similar cheese samples were kept refrigerated (4° C.) for 1 week. Frozen cheeses were thawed overnight in the refrigerator, and all samples were placed at room temperature for 1.5 h before tasting. After this, there was a visual inspection of the cheeses, with attention to color and structure. Textural and taste differences were evaluated by a sensory panel.

### Gouda Jong:

The freeze/thawed cheese is found more rubber-like and dry, when compared to refrigerated cheese. The texture is grainier, crumbling and falls easily apart when applying pressure on the cheese by squeezing the cheese with the fingers or by biting. No consistent flavor defects in the freeze/thawed cheese were identified by the sensory panel.

### Gouda Extra Belegen:

The freeze/thawed cheese seems to be less elastic but creamier compared to the refrigerated cheese. Also the freeze/thawed cheese is grainier, crumbling and falls easily apart when applying pressure on the cheese in comparison to the control. The flavor of the freeze/thawed cheese is described as slightly more salty in the sensory panel, but had less cheese odor.

### Leerdammer Original:

The freeze-thawed cheese was more rubbery compared to the refrigerated cheese. It was more crumbly and has an altered mouthfeel. In the sensory panel, no clear differences between freeze/thawed and refrigerated Leerdammer were found besides the textural changes.

Textural differences were clearly visible between refrigerated and frozen cheeses.

These experiments show that freeze/thawing leads to textural defects in semi-hard continental cheeses

### Example 8: Freezing and Thawing of Pasta Filata Cheese

Mozzarella balls (Galbani 125 g in brine) were either frozen at −20° C. or stored refrigerated at 4° C. for 3 days. After thawing the Mozzarella balls in the refrigerator the cheese was stored another 7 days in the refrigerator. After removal from the refrigerator the texture of the cheeses was manually analyzed by pressing, cutting and tearing. Mozzarella cheese that had been frozen showed a clear deterioration of the fibrous structure compared to non-frozen cheese.

This experiment shows that freezing of pasta filata cheese leads to textural defects when one wishes to store this cheese at refrigerated conditions later on.

### Example 9: Manufacture of Miniature Cheese Using AFP19 from Aspergillus niger

Full fat, non-homogenized, pasteurized milk from Demeter was pre-warmed in a water bath to 25° C. Lactic acid (40% w/v) was added under stirring to adjust the pH of the milk to 6.5. Maxiren 600 (DSM-Food Specialties) was added at 47 IMCU/L and AFP19 was added at 400 nM in the milk before 10 ml of milk are pipetted into each well of a 6 wells microtiter plate. The plate was placed in a 32° C. water bath. After coagulation, the coagulum was cut and then placed into a 38° C. water bath. After 10 minutes, the plate was taken out of the water bath, and a cross shaped magnetic bar (V&P Scientific; Product no #VP772FN-14-34CP) is gently placed into each well. The plate was transferred to a 40° C. incubator with a magnetic levitation stirrer (V&P Scientific). The mixing cycle consisted of mix interval of 30 seconds on/4 minutes off, and the complete mixing cycle was 18 minutes. After the mixing cycle was complete, the plate was centrifuged at 2360 g for 30 minutes in an Eppendorf centrifuge. After centrifugation, the whey was removed, and mini-cheeses were soaked for 30 minutes in a brine solution containing 20% NaCl+0.05% CaCl₂. After this, slices of ˜1 cm in width were individually sealed in a vacuum bag and ripened at 13° C. for 2, 4 or 10 weeks. The cheese was then either directly flash frozen in liquid nitrogen and stored at −80° C., or first frozen and stored for 4 weeks at −20° C., before being flash frozen in liquid nitrogen and stored at −80° C. until further analysis. For each time point and each cheese, two pieces of cheese were frozen, to obtain duplicate measurements.

### Example 10: Microscopic Analysis of Miniature Cheese Prepared in Example 9

A Zeiss LSM710 confocal laser scanning microscope (CSLM) with Axiovert Z1 observer was used for studying the structure of the cheeses.

After defrosting, Fluorescein isothiocyanate (FITC: Sigma-Aldrich) for protein staining and Bodipy® 665/676 (Molecular Probes/Invitrogen) for fat staining, were applied to the cheese. For this, a thin slice of cheese (10×10×1 mm) was placed on a cover slide and 5 μL of a 66 μM Bodipy solution (diluted with EtOH from a 666 μM stock solution in chloroform/ethanol/methanol (1:1:1)) was spread over the cheese. After a few seconds a 5 μL drop of 0.2 mM FITC (diluted with PBS (Sigma-Aldrich) from a 20 mM stock solution in DMSO) was placed on the cheese. After allowing the dyes to be absorbed by the cheese, a second cover slide was placed over the cheese, and the sample was placed with the side of the dyes facing the lens on the CLSM. Several Z-stack images were made of each sample. Both dyes can be visualized simultaneously on the CLSM, as the dye spectra do not overlap.

### Example 11: Image Analysis of Miniature Cheese Prepared in Example 9

An ImageJ plugin (http://imagej.nih.gov/ij/) was developed to measure the 3D volume of non-protein structure (non-green part in the green channel), fat (red particles in the red channel) and holes or cracks (the area which is not stained by green or red). Because a lot more cracks are visible in the slices closest to the microscope objective (may be due to the cutting of the cheese with a scalpel), only the bottom 8 slices (furthest from the objective) were analyzed. For each condition, the cumulative distribution functions (CDF) of crack size (not stained neither in green nor in red part) are compared between ISP (=AFP19) and non-ISP treatments using a Kolmogorov-Smirnov test (KS-test). For the cheeses ripened for 4 and 10 weeks before freezing at −20° C. the average crack size of non-AFP19 treated cheese is significantly larger than the ISP-treated cheese (p<0.005 and p<0.0001 respectively). The average crack size of the different cheeses is depicted in FIG. 1.

A clear increase in crack size becomes apparent in the ripened cheese without AFP19. No such increase in crack size could be detected in cheese that was directly flash frozen in liquid nitrogen without first storage at −20° C. (not shown). Also in the presence of AFP19 no increase in crack size is detected, even when cheeses were frozen at −20° C. (FIG. 1). Apparently the addition of AFP19 in the cheese process prevents deterioration of the cheese structure due to freezing.

These experiments show that AFP19 can prevent textural defects formation upon freezing of cheese

### Example 12: Cream Freeze/Thaw Using AFP19 from Aspergillus niger

AFP19 was added to cream (Campina; 35% fat) at 0, 0.1, 1.0 and 10 mg/l. Addition was 1% (of a concentrated stock solution of AFP19 in water) of the total volume in each. After addition, the samples were gently mixed by stirring. Storage was either for 1 week at 4° C. for the control samples or 1 week at −20° C. Frozen samples were thawed overnight in the refrigerator. All frozen cream samples showed some phase separation after thawing. Samples were stirred gently and the response of the cream on this was monitored. The control cream without added AFP19 and the 0.1 ppm AFP19 sample showed more resistance to stirring and especially the control cream became grainy. In the freeze/thawed samples with 1 and 10 ppm AFP19 the cream is easier to mix and the structure was smooth, almost as smooth as fresh cream.

To investigate the effect of freeze/thawing on the whippability of cream, an additional set of experiments were performed. Cream with 0, 1 and 10 ppm AFP19 was again frozen and thawed. After thawing the cream looked comparable to the cream from the first experiment. The whipping was performed with 100 ml of cream, in a transparent volumetric beaker at a fixed temperature (8±1° C.), and fixed stirring rate, with a hand-held mixer and a fixed time (60 seconds). Volume increase of the freeze/thawed cream after whipping was measured and compared with the refrigerated control.

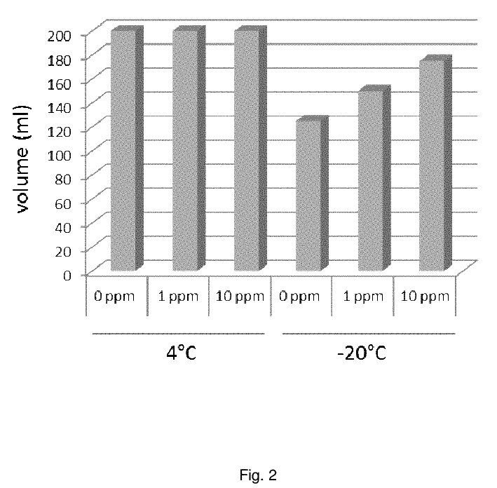

After whipping a volume increase of ˜100% could be established in cream stored at 4° C., irrespective of the amount of AFP19 that was added. After freezing/thawing the volume increase is very low in the frozen control sample upon whipping (˜25% increase in volume). Addition of AFP19 leads to an increase of 50% and 75% in the frozen cream containing 1 and 10 ppm AFP-19 respectively (FIG. 2). The volume of the frozen whipped cream was clearly improved by the addition of AFP-19. The color of all whipped freeze/thawed cream was slightly more yellow. When the whipped freeze/thawed cream was further inspected the look and taste was perceived as more grainy. AFP-19 addition clearly improved the taste perception, especially at 10 ppm, regained much of the smooth texture of non-frozen whipped cream.

These experiments show that AFP19 can prevent textural defects formation and improve whippability of cream that has been freeze/thawed.

### Example 13: Cheddar Cheese Made with AFP19

Cheddar cheese was produced at the pilot plant of the DSM Biotechnologycenter (Delft, the Netherlands) using three 200 liter cheese vats. The cheese vats were filled with 175 liter of full fat, pasteurised bovine milk and tempered to 32° C. Starter culture was added (DelvoTECLL50A; DSM Food-Specialties) at a level of 4 units per 1000 liters of cheese milk. Calcium chloride was added to each vat (50 ml of a 33% solution). After one hour of pre-ripening, rennet (52.5 IMCU/1 Maxiren 600: DSM-Food specialties) was added to the cheese vats and mixed well before being left to stand for the coagulation to take place. Once a firm gel was formed, the coagulum was cut using knives. For 10 minutes the gel was cut using an incremental speed (from 0 to 11). After this time, the cooking step was initiated and in 30 minutes, the temperature of the curd/whey mixture was increased to 38° C. whilst continuous stirring the curd/whey mixture at speed 16. Once this temperature was reached, the curd/whey mixture was stirred until a pH was reached of 6.2 upon which the whey was drained. This was followed by a cheddaring step in which the formed slabs of curd were turned every 15 minutes and stacked after the second turning. Once the pH in the curd slabs had dropped to 5.3, the slabs were milled and split in three portions and dry salted (645 grams of salt were added to the milled curd of 175 liters of full fatmilk). AFP19 was added at 0, 1 and 10 ppm (on total curd weight) mixed with the salt to the different portions. The salted, milled curds were left to mellow for 15 minutes before being transferred to rectangular cheese moulds. The cheeses were pressed overnight at 4 bar. After pressing, two 25 mm thick slab of cheese were cut off with a cheese knife, the thickness was reduced to 20 mm by the aid of an electric deli slicer to ensure an equal thickness of the whole slab. Slabs were vacuum sealed in foil and ripened at 11° C. for 8, 14, 24 and 40 weeks. After ripening the sealed slabs were frozen at −20° C. and stored frozen.

Texture analysis was essentially performed as described previously (O'Callaghan D J, Guinee T P (2004) Rheology and Texture of Cheese. In: Cheese: Chemistry, Physics and Microbiology. 1:511-540).

Before analysis the slabs were defrosted and cylinders of 16 mm width were cut from the slabs with a cheese trier Samples were either stored overnight or for 2 weeks in the fridge at ±4° C. in a ziplock plastic bag. Texture of the cheese samples was tested both by hand and using a TA.HD.plus Texture Analyser (Stable Micro Systems).

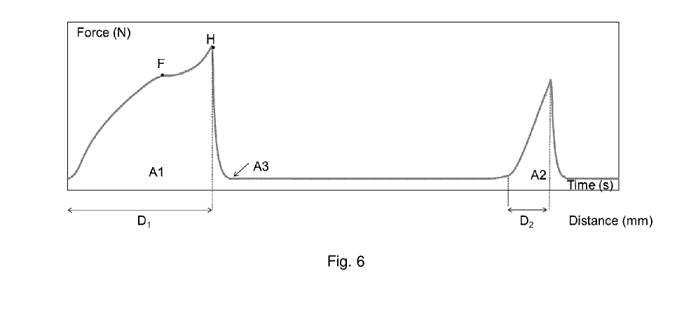

A square compression plate (7×7 cm) was attached to the Texture analyser. For each sample 10 cheese cylinders were analysed and results were averaged. Cheese samples were compressed twice for 30 or 70% at a compression rate of 1 mm/s with 5 seconds waiting time between the two compression cycles (see FIG. 6). The texture analyser started measurement at a trigger force of 0.05 N.

Measurements were recorded with the program Exponent, with this program the peak positive force, peak positive distance, peak force, positive area and negative area of the two peak of each measurement was determined, with Excel the distance at the start of the second peak was determined. From this data the firmness (peak positive force 1), springiness (peak positive distance 2/peak positive distance 1*100%), and cohesiveness (positive area 2/positive area 1) was calculated. A Student's T-test was performed on the data to determine significance of observed differences of the average measurements.

| | | | Calculation |
|---|---|---|---|
| Terminology | Definition | Obtained from | using FIG. 6 |
| Firmness | Force needed to attain a given | Maximum force during first | H (N) |
| | deformation | compression cycle | |
| Springiness | Rate at which deformed food returns to | Percentage of height recovered | D₂/D₁ * 100 (%) |
| | original condition after removal of force | between end of first compression | |
| | | cycle and start of second | |
| Cohesiveness | Strength of food's internal bonds | Ratio of positive force area of | A2/A1 (−) |
| | | second peak to that of first peak | |
| Adhesiveness | Work needed to overcome attractive force | Force area of negative peak (if | Area of A3 |
| | between food and other surface | any) following first peak | (N · sec) |

For firmness measured with TPA no significant difference could be detected between the cheeses of the same age with and without AFP19 added at both 30% and 70% compression. Attributes like cohesiveness and springiness can best be measured at 30% compression in cheese and did show interesting results. Cheeses that had been frozen/thawed were consistently less cohesive and showed a lower springiness than cheeses of the same age that had not been frozen and thawed. Cheeses that had received AFP19 are statistically significant more cohesive and show higher values for springiness after freeze-thaw, and the extend of this effect correlated with the amount of AFP19 that had been added to the cheese, with the cheeses that received 10 ppm AFP19 showed a stronger effect than the cheeses that received 1 ppm AFP19. In fact, the defect in springiness and cohesiveness that was induced by freezing and thawing, was completely absent in the cheeses that had received 10 ppm AFP19.

The same results were obtained by testing the texture of the cheeses by hand: the cheeses containing AFP19 were less crumbly and more cohesive after freeze-thaw than the cheeses lacking the additive. This effect is most clear with the older cheeses.

### Example 14: Whipped Cream

Pasteurized cream (33% fat, 2.5% protein) was bought at a local supermarket (Albert Heijn) and 10% table sugar was added. 100 ml of the cream was whipped using a kitchen mixer for 3 minutes until it was firm. The cream was split in two and either 10 ppm AFP-19 or the same volume of water was mixed well with the whipped cream. The whipped cream was frozen and stored at −18° C. for 3 days.

After defrosting the whipped cream samples were tasted. The sample containing the AFP-19 protein was found clearly more firm by the examiners than the control sample.

1. A method for preparing a food product which food product is frozen and completely thawed before consumption, which method comprises incorporating an ice structuring protein (ISP) in said food product and freezing the prepared food product.

2. A method according to claim 1, further comprising completely thawing of the frozen food product.

3. A food product which is frozen and completely thawed before consumption, wherein said food product comprises an ice structuring protein.

4. A food product obtainable by the method of claim 1.

5. A method according to claim 1 or a food product which is frozen and completely thawed before consumption, wherein said food product comprises an ice structuring protein wherein said ice structuring protein is produced by using a nucleic acid construct which comprises:
a nucleic acid sequence encoding an ice structuring protein (ISP) comprising the sequence set out in SEQ ID NO: 1 or a sequence at least 80% identical thereto or comprising the sequence set out in amino acids 21 to 261 of SEQ ID NO: 1 or a sequence at least 80% identical thereto; and, linked operably thereto,
control sequences permitting expression of the nucleic acid sequence in a filamentous fungal host cell.

6. A method according to claim 1 or a food product which is frozen and completely thawed before consumption, wherein said food product comprises an ice structuring protein wherein said ice structuring protein is produced by a method for the production of an ice structuring protein (ISP), which method comprises:
providing a filamentous fungal host cell which comprises a nucleic acid sequence encoding an ISP comprising the sequence set out in SEQ ID NO: 1 or a sequence at least 80% identical thereto or comprising the sequence set out in amino acids 21 to 261 of SEQ ID NO: 1 or a sequence at least 80% identical thereto,
wherein the said nucleic acid sequence is operably linked to control sequences permitting expression of the nucleic acid sequence in the filamentous fungal host cell;
cultivating the filamentous fungal host cell under conditions suitable for production of the ice structuring protein; and, optionally
recovering the ice structuring protein.

7. A method according to claim 1 or a food product which is frozen and completely thawed before consumption, wherein said food product comprises an ice structuring protein wherein said ice structuring protein comprises the sequence set out in SEQ ID NO: 1 or a sequence at least 80% identical thereto or comprising the sequence set out in amino acids 21 to 261 of SEQ ID NO: 1 or a sequence at least 80% identical thereto, wherein:
at least one amino acid is a modified amino acid;
at least one amino acid is O-mannosylated;
the protein has a glycosylation pattern other than 2GlcNac and 2 hexose units; or
the protein lacks WQKRSNARQWL (SEQ ID NO:4), VQKRSNARQWL (SEQ ID NO:5), or KRSNARQWL (SEQ ID NO:6) at the C-terminus.

8. A food product according to claim 3, which is a water-continuous dispersion.

9. A food product according to claim 3 which is a dairy product, optionally cream or cheese.

10. An ice structuring protein which comprises the sequence set out in SEQ ID NO: 1 or a sequence at least 80% identical thereto or comprising the sequence set out in amino acids 21 to 261 of SEQ ID NO: 1 or a sequence at least 80% identical thereto, wherein:
at least one amino acid is a modified amino acid;
at least one amino acid is O-mannosylated;
the protein has a glycosylation pattern other than 2GlcNac and 2 hexose units; or
the protein lacks WQKRSNARQWL, VQKRSNARQWL or KRSNARQWL at the C-terminus
for preparing a frozen food product which frozen food product is completely thawed before consumption.

11. An ice structuring protein according to claim 10, wherein said frozen food product is a frozen dairy product, optionally frozen cheese or frozen cream.


SEQUENCE LISTING (6 sequences)

SEQ ID NO: 1 - protein, 261 aa, Leucosporidium
MSLLSIITIGLAGLGGLVNGQRDLSVELGVASNFAILAKAGISSVPDSAILGDIGVSPAAATYITGFGLTQDSSTTYATSPQVTGLIYAADYSTPTPNYLAAAVANAETAYNQAAGFVDPDFLELGAGELRDQTLVPGLYKWTSSVSVPTDLTFEGNGDATWVFQIAGGLSLADGVAFTLAGGANSTNIAFQVGDDVTVGKGAHFEGVLLAKRFVTLQTGSSLNGRVLSQTEVALQKATVNSPFVPAPEVVQKRSNARQWL

SEQ ID NO: 2 - DNA, 786 bp, Leucosporidium
  source (1)..(786): /organism=“Leucosporidium” / mol_type=“unassigned DNA”
atgagccttctctccatcatcaccattggtcttgctggcttgggtggtctggtcaacggccagcgtgacctctccgttgagcttggagttgcctccaacttcgccatcctggccaaggccggtatctcctccgtgcctgactctgccatcctcggtgacatcggtgtctctcctgctgctgccacctacatcaccggcttcggtctcacccaggacagcagcaccacctacgccacctcgccccaggtcactggtctcatctacgctgccgactacagcactcccacccccaactacctggctgctgctgttgccaacgccgagactgcctacaaccaggctgctggcttcgtcgaccccgacttcctggagcttggtgctggtgagctgcgtgaccagactctggtgcctggtctctacaagtggacctcctccgtctccgttcctactgacttgaccttcgagggcaacggcgatgccacctgggtgttccagattgctggtggtctctccctcgccgatggtgttgctttcaccctggctggtggtgccaactcgaccaacattgctttccaggtcggcgatgatgtcactgtcggcaagggtgctcacttcgagggtgtcctccttgccaagcgcttcgtcaccctccagactggctcttctctgaacggccgtgtcctgagccagaccgaggttgctctccagaaggccaccgtcaactctcccttcgtccccgctcccgaggttgtgcagaagcgctccaacgcccgccagtggctataa

SEQ ID NO: 3 - DNA, 1059 bp, Artificial sequence
  codon-adapted DNA sequence for expression of SEQ ID NO1 in Aspergillus niger containing additional restriction sites for subcloning in an Aspergillus expression vector
  source (1)..(1059): /organism=“Artificial sequence” /note=“A DNA fragment (comprising the expression cassette as in SEQ ID NO 3) containing a.o. part of the glucoamylase promoter and the ISP encoding gene” /mol_type=“unassigned DNA”
agagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgagccttctctccatcatcaccattggtcttgctggcttgggtggtctggtcaacggccagcgtgacctctccgttgagcttggagttgcctccaacttcgccatcctggccaaggccggtatctcctccgtgcctgactctgccatcctcggtgacatcggtgtctctcctgctgctgccacctacatcaccggcttcggtctcacccaggacagcagcaccacctacgccacctcgccccaggtcactggtctcatctacgctgccgactacagcactcccacccccaactacctggctgctgctgttgccaacgccgagactgcctacaaccaggctgctggcttcgtcgaccccgacttcctggagcttggtgctggtgagctgcgtgaccagactctggtgcctggtctctacaagtggacctcctccgtctccgttcctactgacttgaccttcgagggcaacggcgatgccacctgggtgttccagattgctggtggtctctccctcgccgatggtgttgctttcaccctggctggtggtgccaactcgaccaacattgctttccaggtcggcgatgatgtcactgtcggcaagggtgctcacttcgagggtgtcctccttgccaagcgcttcgtcaccctccagactggctcttctctgaacggccgtgtcctgagccagaccgaggttgctctccagaaggccaccgtcaactctcccttcgtccccgctcccgaggttgtgcagaagcgctccaacgcccgccagtggctataaattaattaacaatcaatccatttcgctatagttaaagga

SEQ ID NO: 4 - protein, 12 aa, Artificial Sequence
  Lacks this sequence at the C-terminus
VVQKRSNARQWL

SEQ ID NO: 5 - protein, 11 aa, Artificial Sequence
  Lacks this sequence at the C-terminus
VQKRSNARQWL

SEQ ID NO: 6 - protein, 9 aa, Artificial Sequence
  Lacks this sequence at the C-terminus
KRSNARQWL